(19) United States
(12) Patent Application Publication
(10) Pub. No.: US 2025/0031953 A1
(43) Pub. Date: Jan. 30, 2025

(54) SYSTEM AND METHOD FOR IN-SITU REMOVAL OF ACCUMULATED MOISTURE AND DEBRIS FROM AN INTRAORAL AMERA

(71) Applicant: Suhaib Harraka, philadelphia, PA (US)

(72) Inventor: Suhaib Harraka, philadelphia, PA (US)

(21) Appl. No.: 18/361,848

(22) Filed: Jul. 29, 2023

(51) Int. Cl. A61B 1/253; A61B 1/00; A61B 1/06; A61B 1/12

(52) U.S. Cl. CPC A61B 1/253; A61B 1/00045; A61B 1/00091; A61B 1/00135; A61B 1/0661; A61B 1/126

(57) ABSTRACT

A system and method are disclosed for in-situ removal of accumulated moisture and debris from a transparent protective cover of a handheld intraoral camera when an oral procedure is performed. The handheld intraoral camera includes a camera unit mounted on an elongated holder, and an elongated sleeve configured to slide over and removably cover the elongated holder and the camera unit. The elongated sleeve includes an opening at a first end for accommodating the transparent protective cover of the camera unit. The elongated sleeve further includes an elongated cavity extending from the opening at the first end of the elongated sleeve to a second end of the elongated sleeve. The system further includes a control unit. The control unit includes a display screen and a compressor for supplying pressurized fluid for the in-situ removal of the accumulated moisture and the debris from the transparent protective cover.

### FIELD OF THE INVENTION

The present invention, in general, relates to an intraoral camera used in the field of dentistry. More specifically, the present invention relates to a system and method for in-situ removal of accumulated moisture and debris from a protective glass cover of a handheld intraoral camera.

### BACKGROUND

The mouth is an oval-shaped cavity inside the skull. The two main functions of the mouth are eating and speaking. Parts of the mouth include the lips, vestibule, mouth cavity, gums, teeth, hard and soft palate, tongue and salivary glands. The mouth is also known as the oral cavity or the buccal cavity.

Humans have used dental instruments for hundreds of years to care for their teeth. Persons primarily use toothbrushes for brushing plaque from teeth, and dental floss for removing food debris stuck between teeth which, if not removed may cause tooth decay and cavities.

Dentists provide services related to the diagnosis, prevention, and treatment of diseases and conditions associated with an oral cavity of a person. Dentists primarily use a variety of instruments, for example, brushes, scalers, curettes, drills, etc., for cleaning and repairing teeth of patients. In use, these instruments are used inside the oral cavity of the patient to clean, restore or repair teeth and tissue in an oral cavity.

However, it is often difficult for a dentist to clearly see inside a patient's mouth. More specifically, it is often difficult to open the patient's mouth wide enough to allow the dentist to place an instrument or instruments in the patient's mouth and see inside the patient's mouth. In many cases, work performed within an oral cavity can only be viewed at uncomfortable angles depending on where the work is being performed.

To overcome the above problems, dentists often use an intraoral camera to view the oral cavity of the patient. The intraoral camera may be mounted at the end of a pole and inserted in the oral cavity of the patient to view the space in the oral cavity. However, such intraoral cameras are often used concurrently with and separately from dental instruments used to work on teeth and/or tissue within the oral cavity. Oftentimes, debris for example, dental plaque, food particles, tartar, ground tooth particles, etc., may get deposited on a lens or transparent protective cover of the intraoral camera. The lens or transparent protective cover of the intraoral camera may also get fogged during use in the oral cavity of the patient's mouth. Therefore, it becomes difficult for the dentist to efficiently use the intraoral camera as needed and for as long as required to view the region of the oral cavity that is being worked on by the dental instrument. A dentist may be required to manipulate both the intraoral camera and the dental instrument to dislodge the debris from the lens or the transparent protective cover of the intraoral camera.

To clean the transparent protective cover of the intraoral camera, the intraoral camera may be required to be removed from the patient's mouth and thereafter the transparent protective cover manually wiped clean. Therefore, there is a long felt yet unresolved need for an intraoral camera comprising an integrated apparatus for cleaning the transparent protective cover when the intraoral camera is inside the patient's mouth during an oral procedure. There is also a long felt yet unresolved need for an intraoral camera comprising an integrated apparatus that allows a hands-free cleaning of the transparent protective cover of the intraoral camera.

### SUMMARY OF THE INVENTION

This summary is provided to introduce a selection of concepts in a simplified form that are further disclosed in the detailed description of the invention. This summary is not intended to determine the scope of the claimed subject matter.

A system and method are disclosed for in-situ removal of accumulated moisture and debris from a transparent protective cover of a handheld intraoral camera disposed at a procedural site within an oral cavity or mouth of a patient when an oral procedure is performed. The system comprises a handheld intraoral camera and a control unit.

The handheld intraoral camera comprises a camera unit mounted on an elongated holder and an elongated sleeve. The elongated sleeve is configured to slide over and removably cover the elongated holder and the camera unit. The elongated sleeve comprises an opening at a first end for accommodating the transparent protective cover of the camera unit. The elongated sleeve further comprises an elongated cavity extending from the opening at the first end of the elongated sleeve to a second end of the elongated sleeve. The elongated cavity comprises a nozzle proximal to the opening at the first end of the elongated sleeve. The nozzle is configured to discharge a pressurized fluid, for example, pressurized air, water, or a combination thereof.

The control unit comprises a display screen and a compressor. The display screen displays images of the procedural site when the oral procedure is performed. The compressor provides pressurized fluid for the in-situ removal of the accumulated moisture and debris from the transparent protective cover of the handheld intraoral camera disposed at the procedural site within the mouth of the patient when the oral procedure is performed. The transparent protective cover is made of either glass, plastic, quartz, etc. As used herein the terms mouth and oral cavity are used interchangeably.

The method comprises inserting the handheld intraoral camera into the procedural site within the mouth of the patient and obtaining an image of the procedural site on the display screen of the control unit.

When the obtained image is blurred due to the accumulation of moisture and debris on the transparent protective cover of the handheld intraoral camera, the user, for example, the dentist operates the compressor to blow the pressurized fluid on to the transparent protective cover of the camera unit. The pressurized fluid, for example, pressurized air, pressurized water, or a combination thereof may also be blown on to the procedural site. The pressurized fluid removes the accumulated moisture and debris from the transparent protective cover of the handheld intraoral camera when the oral procedure is performed.

### BRIEF DESCRIPTION OF THE DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIGS. 1A and 1B illustrate front top perspective views of a handheld intraoral camera comprising an elongated sleeve.

FIG. 2 illustrates a front top perspective view of the handheld intraoral camera with the elongated sleeve removed from the intraoral camera to show an elongated holder comprising a camera unit.

FIG. 3A illustrates another top perspective view of the handheld intraoral camera with the elongated sleeve removed to show the elongated holder comprising the camera unit.

FIG. 3B illustrates a top perspective view of the elongated holder comprising the camera unit.

FIG. 3C illustrates a top perspective view of the elongated sleeve.

FIG. 3D illustrates a cross-sectional view of the elongated sleeve taken along line D-D′ shown in FIG. 3C.

FIG. 4 illustrates a close-up view of a transparent protective cover of a camera unit of the handheld intraoral camera.

FIG. 5 illustrates another close-up view of the transparent protective cover of the camera unit of the handheld intraoral camera.

FIG. 6A illustrates a rear view of an embodiment of a system for in-situ removal of accumulated moisture from a transparent protective cover of the handheld intraoral camera disposed at a procedural site within mouth of a patient when an oral procedure is performed.

FIG. 6B illustrates a front view of the embodiment of the system shown in FIG. 6A illustrating a display screen showing a feed from the handheld intraoral camera and a foot pedal used to control burst of pressurized fluid supplied to an elongated cavity in the elongated sleeve.

FIG. 7A illustrates a rear view of another embodiment of the system for in-situ removal of accumulated moisture from the transparent protective cover of the handheld intraoral camera.

FIG. 7B illustrates a front view of the embodiment of the system shown in FIG. 7A illustrating a display screen showing a feed from the handheld intraoral camera and two foot pedals used to control bursts of pressurized air and pressurized water supplied to an elongated cavity in the elongated sleeve.

FIG. 8 illustrates a method for in-situ removal of accumulated moisture from a transparent protective cover of a handheld intraoral camera disposed at a procedural site within the mouth of a patient when an oral procedure is performed.

### DETAILED DESCRIPTION OF THE INVENTION

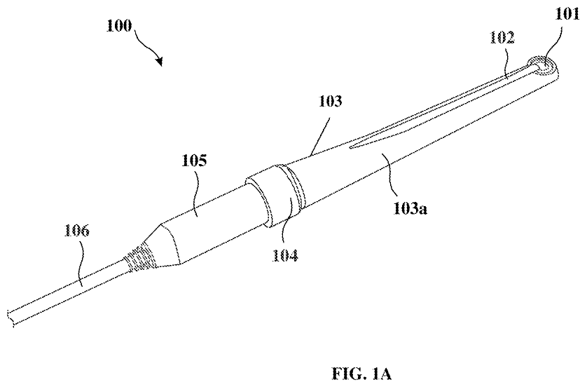

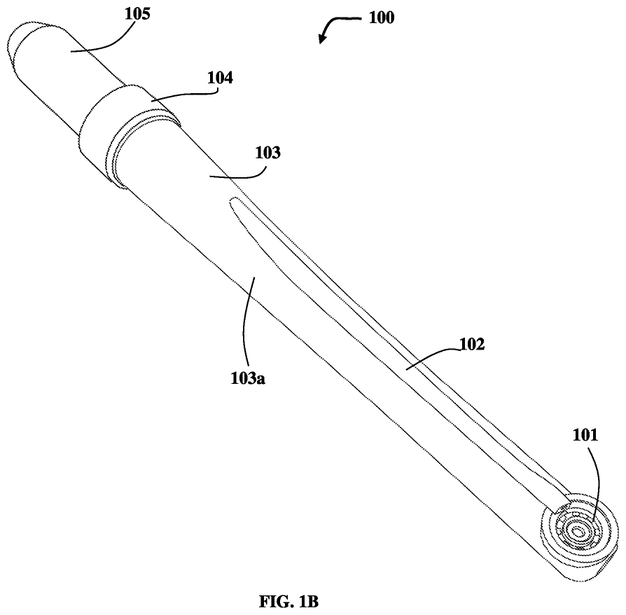

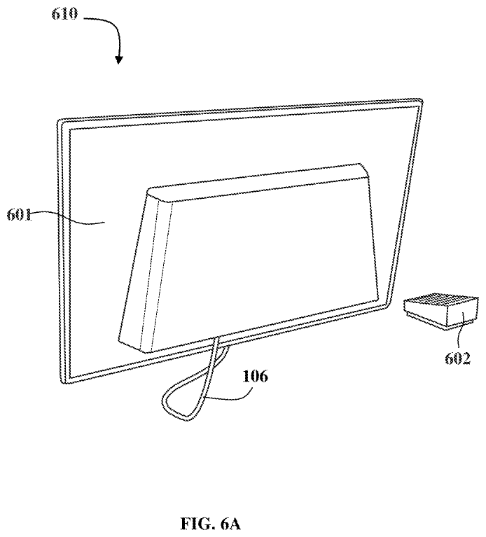

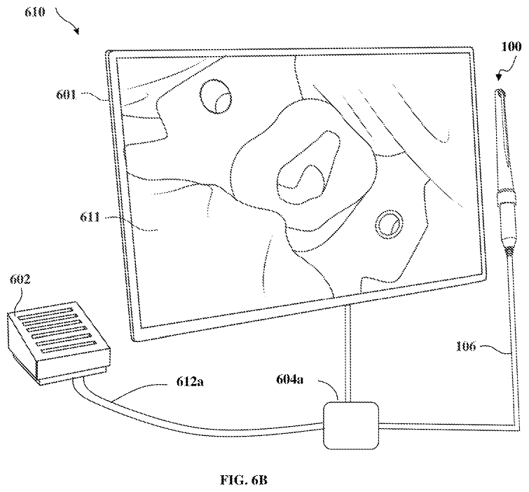

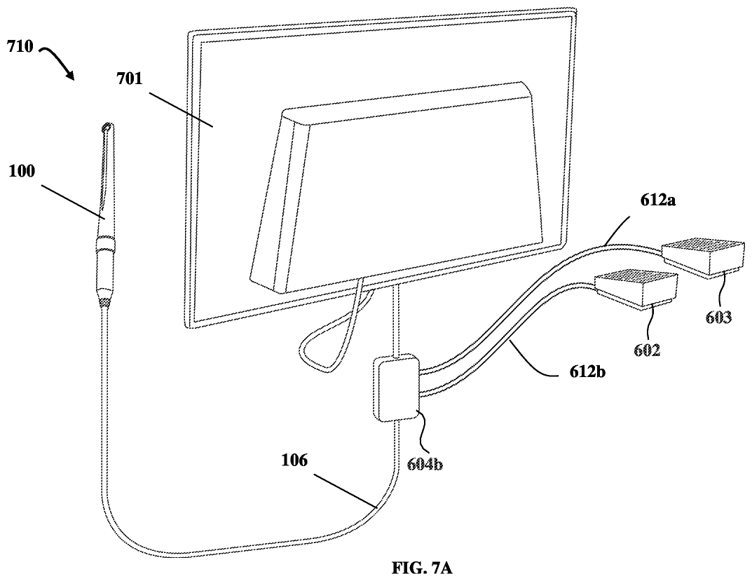

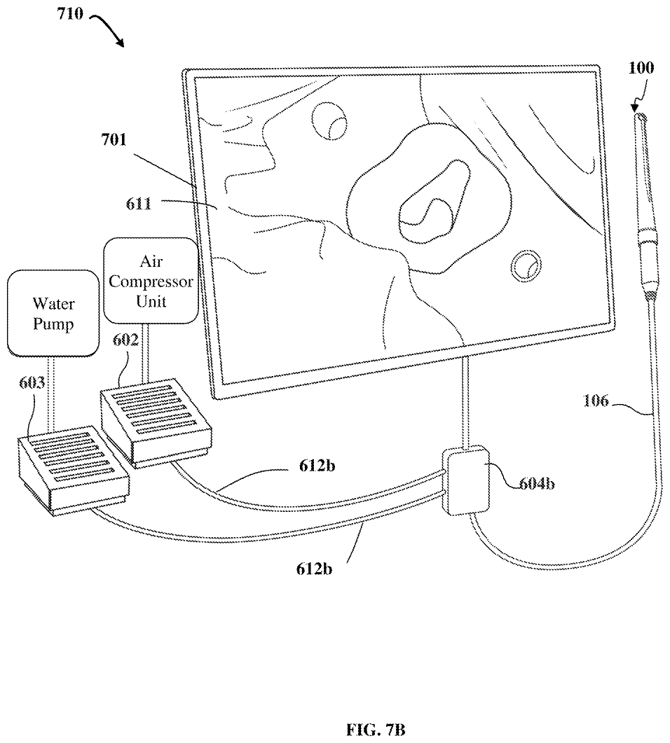

FIGS. 6A-7B illustrate embodiments of a system 610, 710 for in-situ removal of accumulated moisture from a transparent protective cover 101 of a handheld intraoral camera 100, shown in FIG. 1A, disposed at a procedural site within the mouth of a patient when an oral procedure is performed. The systems 610, 710 comprises the handheld intraoral camera 100 and a control unit 601 or 701, shown in FIGS. 6A-7B.

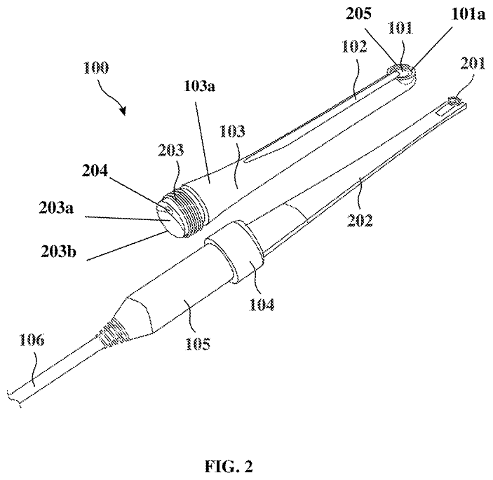

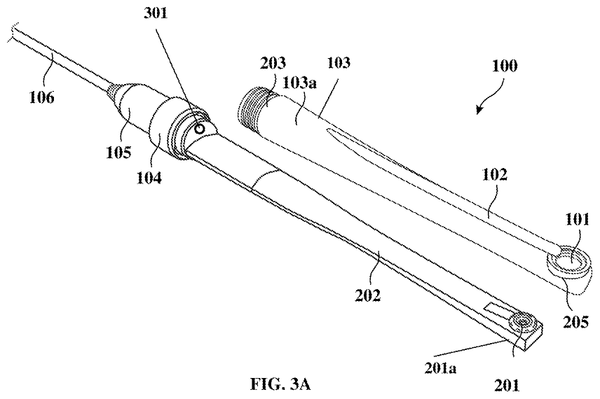

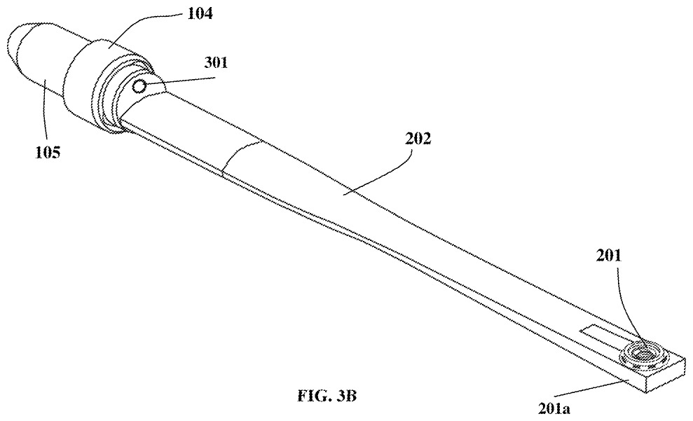

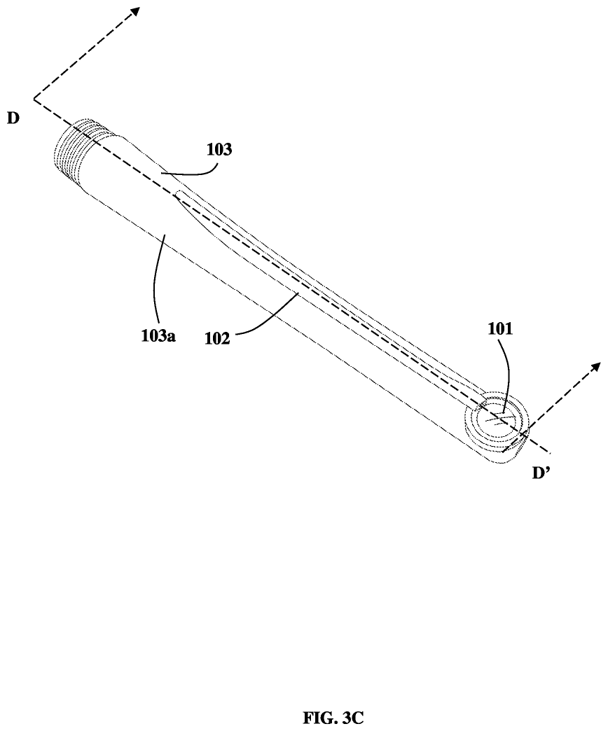

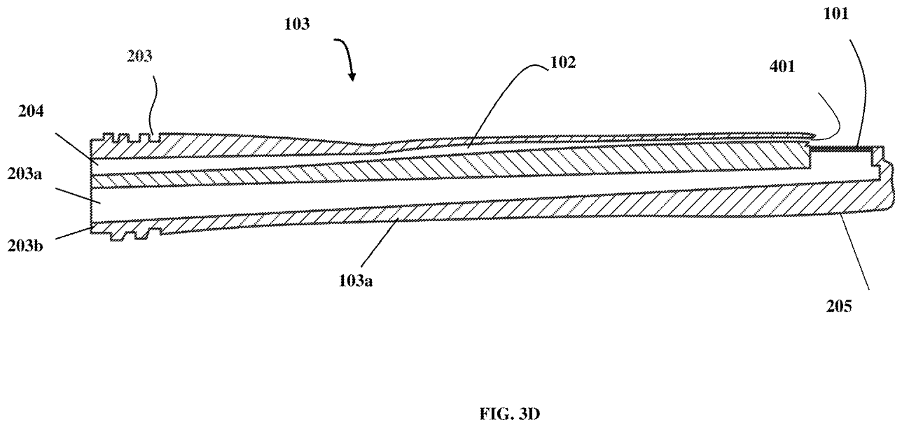

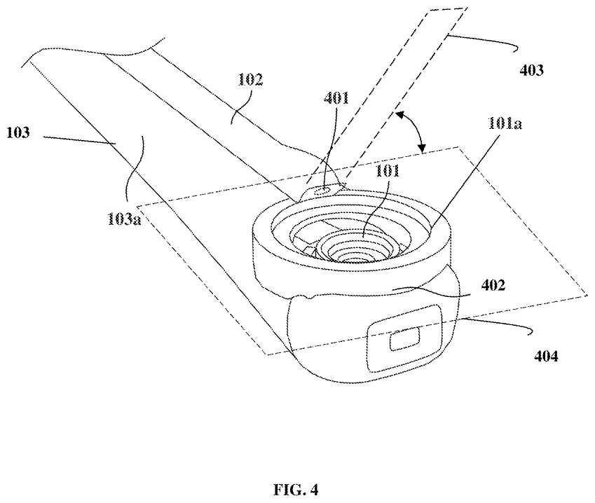

FIGS. 1A and 1B illustrate front top perspective views of the handheld intraoral camera 100 comprising an elongated sleeve 103. The handheld intraoral camera 100 allows dentists to obtain a magnified image or video of the tooth, the gums, or any procedural site within the mouth, and to perform oral procedures effectively. The handheld intraoral camera 100 comprises a camera unit 201, shown in FIGS. 2, 3A, and 3B mounted on an elongated holder 202, and an elongated sleeve 103. The elongated sleeve 103 is configured to slide over and removably engage with the elongated holder 202 and the camera unit 201. The elongated sleeve 103 comprises an opening 101a at a first end 205, as shown in FIG. 4, for accommodating the transparent protective cover 101 of the camera unit 201. The elongated sleeve 103 comprises a cavity 203a that extends from a second end 203 of the elongated sleeve 103 to a first end 205 of the elongated sleeve 103. The cavity 203a comprises an opening 203b for receiving the elongated holder 202, more particularly the end 201a of the elongated holder 202 comprising the camera unit 201. In an embodiment, the entire length of the elongated holder 202 is accommodated in the elongated sleeve 103. The transparent protective cover 101 is made of glass, plastic, quartz, etc. The transparent protective cover 101 and the elongated sleeve 103 enables a dentist to insert the camera unit 201 into the mouth of a patient and capture images and/or video of the tooth, gums, and other procedural site within the mouth of the patient without affecting the operation of the camera unit 201. The portion of the handheld intraoral camera 100 at the first end 205 of the elongated sleeve 103 is inserted into the mouth of the patient.

In an embodiment, the elongated sleeve 103 is made from a metallic material, to withstand head of a pump that supplies pressurized fluid to the elongated sleeve 103. Furthermore, the elongated sleeve 103 is made to withstand the pressure exerted by the pressurized fluid on the elongated sleeve 103. The elongated sleeve 103 may be cleaned using soap, disinfectant, and/or using an autoclave after inserting the elongated sleeve 103 in a patient's mouth.

The elongated sleeve 103 further comprises an elongated cavity 102, as shown in FIG. 3D, extending from an opening 401, shown in FIGS. 3D and 4, proximal to the first end 205 of the elongated sleeve 103 to an opening 204 at a second end 203 of the elongated sleeve 103. The opening 401 is shaped in the form of a nozzle 401, as shown in FIGS. 3D and 4. The nozzle 401 is configured to discharge a pressurized fluid, for example, pressurized air, pressurized water, or a combination thereof. Nozzle 401 is disposed proximal to the transparent protective cover 101. Pressurized fluid, for example, pressurized air, pressurized water, etc., is directed continuously and in bursts towards the transparent protective cover 101 while an oral procedure is performed to remove moisture and debris from the transparent protective cover 101. The nozzle 401 is in fluid communication with a compressor, for example, an air compressor unit, shown in the embodiment of the system 710 and the control unit 701 illustrated in FIG. 7B. The compressor is controlled by a foot pedal 602, 603, as shown in FIGS. 6A-7B.

As shown in FIGS. 1A-3A and 3C-5, the elongated cavity 102 is located on an outer surface 103a of the elongated sleeve 103. Although the elongated cavity 102 is located on the outer surface 103a of the elongated sleeve 103, in an embodiment, the elongated cavity 102 is integrated to the body of the elongated sleeve 103. The cavity opening 204, shown in FIG. 2, located at the second end 203 of the elongated sleeve 103 is configured to receive the pressurized fluid from the compressor, for example, the air compressor unit, shown in the embodiment of the system 710 illustrated in FIG. 7B.

FIG. 2 illustrates a top perspective view of the handheld intraoral camera 100 with the elongated sleeve 103 removed from the intraoral camera to show an elongated holder comprising a camera unit. The handheld intraoral camera 100 further comprises a handle 105, shown in FIG. 1A, configured to be attached to the elongated holder 202. The handle 105 comprises an interface 106 for connecting to the camera unit 201 through the elongated holder 202. The interface 106 comprises a signal line (not shown) for transferring images from the camera unit 201 to a display screen 611 via a coupler 604a, shown in FIGS. 6A-7B, of a control unit 601, 701. In this embodiment, the coupler merely binds the signal line (not shown) and the hose 612a to create the interface 106. The interface 106 comprises the signal line (not shown) and a passage (not shown). The passage is in fluid connection with the hose 612a to allow transfer of the pressurized fluid from the compressor to the nozzle 401 of the elongated cavity 102 of the elongated sleeve 103. The interface 106 allows no contact between the signal line (not shown) and the pressurized fluid carried by the passage (not shown).

The handle 105 is detachably attached to the second end 203, shown in FIG. 3A, of the elongated sleeve 103 using a fastener 104. The fastener is, for example, a ring, as shown in FIGS. 1A-3S. The ring 104 is screwed and unscrewed to attach and detach respectively the handle 105 to the elongated sleeve 103 of the handheld intraoral camera 100. In an embodiment, the handle 105 is integrated to the elongated holder 202 comprising the camera unit 201. In another embodiment, the handle 105 is detachably attached to the elongated holder 202 comprising the camera unit 201. The ring 104 is internally threaded and the second end 203 of the elongated sleeve 103 is externally threaded, as shown in FIGS. 2 and 3A. The second end 203 of the elongated sleeve 103 is inserted into the ring 104 and the ring 104 is threaded onto the second end 203 of the elongated sleeve 103.

FIG. 3A illustrates another top perspective view of the handheld intraoral camera 100 with the elongated sleeve 103 removed to show the elongated holder 202 comprising the camera unit 201. FIG. 3B illustrates a top perspective view of the elongated holder 202 comprising the camera unit 201. FIG. 3C illustrates a top perspective view of the elongated sleeve 103. FIG. 3D illustrates a cross-sectional view of the elongated sleeve 103 taken along line D-D′ shown in FIG. 3C. The ring 104 is unscrewed to detach the elongated sleeve 103 from the handle 105. The interface 106 further comprises at least one passage (not shown) for transferring pressurized fluid from the compressor, for example, an air compressor, shown in FIG. 7B, to the nozzle 401 of the elongated cavity 102 via a channel 301 in the elongated holder 202, as shown in FIGS. 3A and 3B. In an embodiment, the pressurized fluid is transferred in short bursts to the nozzle 401 through the channel 301 and the elongated cavity 102. The channel 301 and the elongated cavity 102 are substantially aligned with each other to facilitate passage of the pressurized fluid from the compressor to the nozzle 401. As used herein, the pressurized fluid is one of pressurized air, pressurized water, and a combination thereof.

FIG. 4 illustrates a close-up view of a transparent protective cover 101 of the camera unit 201 of the handheld intraoral camera 100. As shown in FIGS. 3D and 4, the nozzle 401 at the first end 205 of the elongated sleeve 103 is angled towards the transparent protective cover 101 of the camera unit 201 to effectively direct the discharge of the pressurized fluid towards the transparent protective cover 101. As shown in FIG. 4, an angle between a plane 403 of the nozzle 401 and a plane of the transparent protective cover 101 is an acute angle.

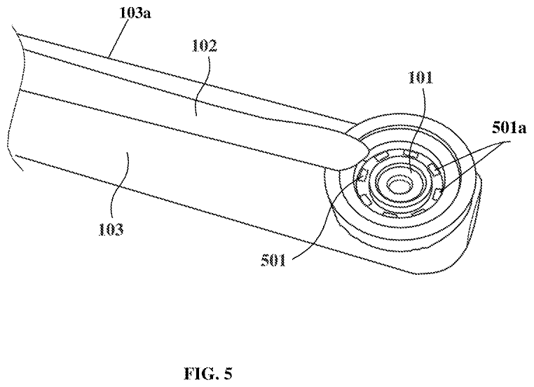

FIG. 5 illustrates another close-up view of the transparent protective cover 101 of the camera unit 201 of the handheld intraoral camera 100. As shown in FIGS. 3-5, the camera unit 201 further comprises an integrated light source 501, shown in FIG. 5, for providing illumination. The light source 501 is, for example, a LED ring, comprising multiple LEDs 501a arranged in the form of a ring, as shown in FIG. 5.

FIG. 6A illustrates a rear view of an embodiment of a system 610 for in-situ removal of accumulated moisture from a transparent protective cover 101 of the handheld intraoral camera 100 disposed at a procedural site within mouth of a patient when an oral procedure is performed. FIG. 6B illustrates a front view of the embodiment of the system shown in FIG. 6A illustrating a display screen 611 showing a feed from the handheld intraoral camera 100 and a foot pedal 602 used to control burst of pressurized fluid supplied to an elongated cavity 102 in the elongated sleeve 103. As shown in FIGS. 6A and 6B, the system 610 comprises a control unit 601 and a foot pedal 602. The control unit 601 comprises the display screen 611 and a compressor, for example, an air compressor, shown in FIG. 7B. The display screen 611 displays images of the procedural site when the oral procedure is performed, as shown in FIG. 6B. The compressor is integrated into the control unit 601 for providing pressurized fluid, for example, pressurized air for the in-situ removal of the accumulated moisture and debris from the transparent protective cover 101 of the handheld intraoral camera 100 disposed at the procedural site within the mouth of the patient when the oral procedure is performed. This facilitates a clear view of the procedural site to a dentist while performing the oral procedure.

FIG. 7A illustrates a rear view of another embodiment of the system 710 for in-situ removal of accumulated moisture from the transparent protective cover 101 of the handheld intraoral camera 100. FIG. 7B illustrates a front view of the embodiment of the system 710 shown in FIG. 7A illustrating a display screen 611 showing a feed from the handheld intraoral camera 100 and two foot pedals 602 and 603 used to control bursts of pressurized air and pressurized water supplied to an elongated cavity 102 in the elongated sleeve 103. As shown in FIGS. 7A and 7B, the system comprises two foot pedals 602 and 603, and a coupler 604b. The first foot pedal 602 is used to control bursts of pressurized air supplied from the air compressor unit to the elongated cavity 102 in the elongated sleeve 103, and the second foot pedal 603 is used to control bursts of pressurized water supplied from the water pump to the elongated cavity 102 in the elongated sleeve 103. An operator, for example, a dentist may operate one or both of the foot pedals 602 and 603 at a time to deliver either pressurized air, pressurized water, or a combination of pressurized air and pressurized water to the elongated cavity 102 in the elongated sleeve 103. The coupler 604b is used to link together the hose 612a that carries pressurized air and the hose 612b that carries pressurized water. The interface 106 comprises the signal line (not shown) and a passage (not shown). The passage (not shown) of the interface 106 is also connected to the coupler 604b to transfer the pressurized fluid, for example, the pressurized air, pressurized water, or a combination of pressurized air and pressurized water, from the hose 612a and the hose 612b, to the nozzle 401 of the elongated cavity 102 via the channel 301 shown in FIGS. 3A and 3B. The interface 106 allows no contact between the signal line (not shown) and the pressurized fluid carried by the passage (not shown).

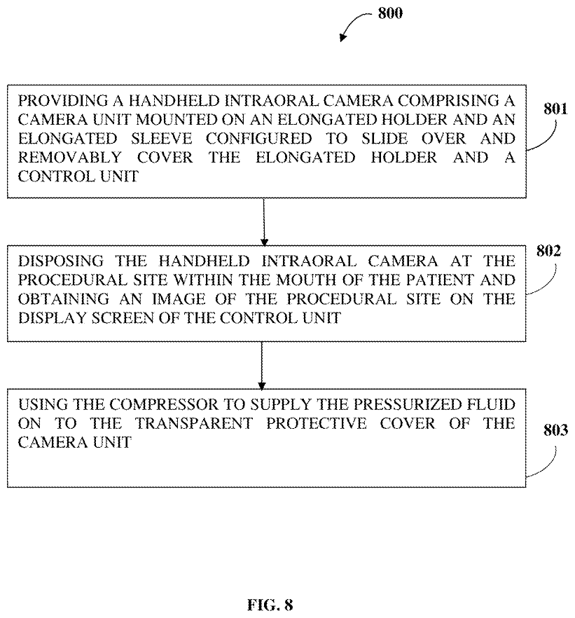

FIG. 8 illustrates a method 800 for in-situ removal of accumulated moisture from a transparent protective cover 101 of a handheld intraoral camera 100 disposed at a procedural site within the mouth of a patient when an oral procedure is performed.

The method 800 comprises inserting the handheld intraoral camera 100 into the procedural site within the mouth of the patient and obtaining an image of the procedural site on the display screen 611 of the control unit 601.

The method further comprises using the compressor to blow pressurized fluid on the transparent protective cover 101 of the camera unit 201 when the obtained image and/or video is blurred due to accumulation of moisture and debris on the transparent protective cover 101 of the camera unit 201 of the handheld intraoral camera 100. The compressor and/or water pump is operated to supply the pressurized fluid on to the transparent protective cover 101, through the nozzle 401 in the elongated cavity 102, to remove the accumulated moisture and debris from the transparent protective cover 101 of the handheld intraoral camera 100 when the oral procedure is performed. In an embodiment, the pressurized fluid is blown on the procedural site for removing the accumulated moisture and debris from the procedural site.

The foregoing examples have been provided merely for explanation and are in no way to be construed as limiting of the method 800 and the systems 610, 710 disclosed herein. While the method 800 and the system 610, 710 have been described with reference to various embodiments, it is understood that the words, which have been used herein, are words of description and illustration, rather than words of limitation. Furthermore, although the method 800 and the system 610, 710 have been described herein with reference to particular means, materials, and embodiments, the method 800 and the system 610, 710 are not intended to be limited to the particulars disclosed herein; rather, the method 800 and the system 610, 710 extend to all functionally equivalent structures, methods and uses, such as are within the scope of the appended claims. While multiple embodiments are disclosed, it will be understood by those skilled in the art, having the benefit of the teachings of this specification, that the method and the system 610 disclosed herein are capable of modifications and other embodiments may be effected and changes may be made thereto, without departing from the scope and spirit of the method 800 and the system 610, 710 disclosed herein.

I claim:

1. A system for in-situ removal of accumulated moisture and debris from a transparent protective cover of a handheld intraoral camera disposed at a procedural site in a mouth of a patient when an oral procedure is performed, comprising:
said handheld intraoral camera, comprising:
a camera unit mounted on an elongated holder; and
an elongated sleeve configured to slide over and removably cover the elongated holder and the camera unit, wherein the elongated sleeve comprises an opening at a first end for accommodating the transparent protective cover of the camera unit, and wherein the elongated sleeve further comprises:
an elongated cavity extending from the opening at the first end of the elongated sleeve to a second end of the elongated sleeve,
wherein the elongated cavity comprises a nozzle proximal to the opening at the first end of the elongated sleeve; and
a control unit, comprising:
a display screen for displaying images of the procedural site when the oral procedure is performed; and
a compressor for providing pressurized fluid, wherein the pressurized fluid travels from the compressor to the nozzle of the elongated cavity via a channel in the elongated holder for the in-situ removal of the accumulated moisture and the debris from the transparent protective cover of the handheld intraoral camera disposed at the procedural site within the mouth of the patient when the oral procedure is performed.

2. The system of claim 1, wherein the handheld intraoral camera further comprises a handle configured to be attached to the elongated holder, wherein the handle comprises an interface for connecting the camera unit through the elongated holder, and wherein the interface comprises a signal line for transferring images from the camera unit to the display screen of the control unit, and wherein the interface further comprises a passage for delivering the pressurized fluid from the compressor to the nozzle of the elongated cavity.

3. The system of claim 2, wherein the handle of the handheld intraoral camera is detachably attached to the elongated sleeve of the handheld intraoral camera using a fastener.

4. The system of claim 3, wherein the fastener is a ring that is screwed and unscrewed to attach and detach respectively the handle to the elongated sleeve of the handheld intraoral camera.

5. The system of claim 1, wherein the camera unit of the handheld intraoral camera further comprises an integrated light source for providing illumination to the procedural site when the procedure is performed.

6. The system of claim 5, wherein the integrated light source is a ring light.

7. The system of claim 2, wherein flow of the pressurized fluid to the elongated cavity is controlled by a foot pedal.

8. The system of claim 1, wherein the fluid is one or more of air and water, and wherein the elongated sleeve is made from a metallic material, and wherein the elongated sleeve is configured to be cleaned using one of an autoclave, a soap, and a disinfected after insertion into a patient's mouth.

9. A method for in-situ removal of accumulated moisture and debris from a transparent protective cover of a handheld intraoral camera disposed at a procedural site within a mouth of a patient when an oral procedure is performed, comprising:
providing the handheld intraoral camera, comprising:
a camera unit mounted on an elongated holder; and
an elongated sleeve configured to slide over and removably cover the elongated holder and the camera unit, wherein the elongated sleeve comprises an opening at a first end for accommodating a transparent protective cover of the camera unit, and wherein the elongated sleeve further comprises:
an elongated cavity extending from the opening at the first end of the elongated sleeve to a second end of the elongated sleeve,
wherein the elongated cavity comprises a nozzle proximal to the opening at the first end of the elongated sleeve; and
a control unit, comprising:
a display screen for displaying images of the procedural site when the oral procedure is performed; and
a compressor for providing pressurized fluid;
disposing the handheld intraoral camera at the procedural site within the mouth of the patient and obtaining an image of the procedural site on the display screen of the control unit; and
using the compressor to supply the pressurized fluid, wherein the pressurized fluid travels from the compressor to the nozzle of the elongated cavity via a channel in the elongated holder for the in-situ removal of the accumulated moisture and the debris from the transparent protective cover of the handheld intraoral camera disposed at the procedural site within the mouth of the patient when the oral procedure is performed.

10. The method of claim 9, further comprises blowing the pressurized fluid to the procedural site.

11. The method of claim 9, wherein the pressurized fluid is one or more of air and water.

12. The method of claim 9 further comprises:
attaching a handle to the elongated holder, wherein the handle comprises an interface for connecting the camera unit through the elongated holder; and
transferring images from the camera unit to the display screen of the control unit through a signal line of the interface, wherein the interface further comprises a passage for delivering the pressurized fluid from the compressor to the nozzle of the elongated cavity.

13. A handheld intraoral camera, comprising:
a camera unit mounted on an elongated holder; and
an elongated sleeve configured to slide over and removably cover the elongated holder and the camera unit, wherein the elongated sleeve comprises an opening at a first end for accommodating the transparent protective cover of the camera unit, and wherein the elongated sleeve further comprises:
an elongated cavity extending from the opening at the first end of the elongated sleeve to a second end of the elongated sleeve, wherein the elongated cavity comprises a nozzle proximal to the opening at the first end of the elongated sleeve.

14. The handheld intraoral camera of claim 13, further comprising a handle configured to be attached to the elongated holder, wherein the handle comprises an interface for connecting to the camera unit through the elongated holder, wherein the interface comprises a signal line for transferring images from the camera unit to a display screen of a control unit, and wherein the interface further comprises a hose for transferring pressurized fluid from a compressor in the control unit to the nozzle of the elongated cavity.

15. The handheld intraoral camera of claim 14, wherein the handle is detachably fastened to the elongated sleeve using a fastener.

16. The method of claim 15, wherein the fastener is a ring that is screwed and unscrewed to attach and detach respectively the handle to the elongated sleeve of the handheld intraoral camera.

17. The handheld intraoral camera of claim 13, wherein the camera unit further comprises an integrated light source for providing illumination.

18. The handheld intraoral camera of claim 17, wherein the integrated light source is a ring light.

19. The handheld intraoral camera of claim 14, wherein flow of the pressurized fluid to the elongated cavity is controlled by a foot pedal.

20. The handheld intraoral camera of claim 13, wherein the elongated sleeve is made from a metallic material, and wherein the elongated sleeve is configured to be cleaned using one of an autoclave, a soap, and a disinfected after insertion into a patient's mouth.
